(19) United States
(12) Patent Application Publication
(10) Pub. No.: US 2019/0151580 A1
(43) Pub. Date: May 23, 2019

(54) VENTILATOR WITH FUNCTIONAL MODULE

(71) Applicant: AUPU Home Style Corporation Limited., Hangzhou (CN)

(72) Inventors: Shouyong GAO, Hangzhou (CN); Jian CHEN, Hangzhou (CN); Junjie TANG, Hangzhou (CN); Jianli XU, Hangzhou (CN); Zhe LI, Hangzhou (CN)

(21) Appl. No.: 16/180,929

(22) Filed: Nov. 5, 2018

(30) Foreign Application Priority Data
Nov. 22, 2017 (CN) 201721577530.8

(51) Int. Cl. A61M 16/00

(52) U.S. Cl. CPC A61M 16/0003; A61M 2205/3327; A61M 2205/3303

(57) ABSTRACT

The utility model relates to a ventilator with a functional module, belonging to the technical field of ventilator equipment. Customers cannot add or reduce functions in existing ventilator products, which lowers the flexibility of use, generates waste or fails to meet demands, and also increases production cost. In the utility model, a functional component is of an independent modular design: the functional component is independently placed in the module case to become a functional module such that the function can be customized according to demands. The utility model adopts the common micro-USB as a connecting terminal, and the ventilator body is provided with a plurality of micro-USB receptacles as connecting ports, realizing modular plug-and-pull and ensuring random plug-and-pull. Thus, the utility model is simple in connection operation, ensures plug-and-pull, is easy to identify, avoids mis-connection, and increases safety.

### BACKGROUND OF THE INVENTION

### Technical Field

The utility model relates to a ventilator with a functional module, belonging to the technical field of ventilator equipment.

### Description of the Related Art

Besides the main ventilation function, existing ventilator products usually have other additional functions such as a human body sensing function, humidity detection function, night-light function, etc. Those working functional components are mainly integrated into the product and sold together with the product, so that the additional functions are fixed, and customers cannot add or reduce the relevant functional modules, thus lowering the flexibility of use, generating waste or failing to meet demands, and adding production costs at the same time. In addition, components are in a one-to-one connection, and one functional component can correspond to only one connecting port. Customers need to find corresponding ports when changing and installing functional components. Doing so will cause the risk of mis-plugging the connecting port, thus generating potential safety hazards such as component failure, short circuits, and product damage. During replacement, existing products are usually connected using connectors such as screws, bringing inconvenience in dismantling and installation.

### BRIEF SUMMARY OF THE INVENTION

Aiming at defects in the prior art, the objective of the utility model is to provide a ventilator with a functional module, which can be flexibly added with functional modules, is convenient in installation and low in cost, and can meet the various demands of customers.

To achieve the above object, the utility model adopts the following technical scheme:

A ventilator with a functional module includes a ventilator body; the ventilator body is connected with a functional module; the functional includes a module case capable of accommodating a functional component, and a connecting wire; the module case is in a buckled connection with the ventilator body; the ventilator body is provided with a plurality of micro-USB receptacles; one end of the connecting wire is connected with the functional component, and the other end is connected with one of the micro-USB receptacles through a micro-USB terminal.

In the utility model, the functional component is of an independent modular design: the functional component is independently placed in the module case to become a functional module such that the component can be separated from or assembled with the product and the function can be customized according to demands, thus increasing the diversity of the product functions and the flexibility of customer selections. The module case is of a fastening design, and does not need other connecting parts such as screws during installation. The modular installation is more convenient, and cost is lower.

The utility model adopts the common micro-USB as a connecting terminal, and the ventilator body is provided with a plurality of micro-USB receptacles as connecting ports, realizing modular plug-and-pull and ensuring random plug-and-pull. Thus, the utility model is simple in connection operation, ensures plug-and-pull, is easy to identify, avoids mis-connection, and increases safety.

As a preferred technical means, the connecting wire is provided with a common terminal which is fixedly connected with an output interface of the functional component, and a data wire capable of transmitting data is connected between the common terminal and the micro-USB terminal. The length of the data wire can be flexibly set according to demands so that the installation position of the module case is more flexible, and the shape of the data wire may be round or square.

As a preferred technical means, the data wire penetrates through a wire clip, and the wire clip is placed in an inner cavity of the module case. The module case is provided with an outgoing port; the data wire passes through the outgoing port, thus preventing the data wire from being pulled and falling out of the module case by an external force, and increasing the reliability of the use of the connecting wire.

As a preferred technical means, the module case is provided with a plurality of fixed buckles; the ventilator body is provided with a plurality of corresponding buckling positions; the fixed buckles of the module case and the buckling positions on the ventilator body are mutually buckled to complete the fixed installation of the module case and the ventilator body. Due to the buckle position structure, connectors such as screws are reduced, time is saved during installation or dismantling, and the installation becomes simpler and more convenient.

As a preferred technical means, the module case is of a cavity structure, and has an appearance shape matched with the appearance shape of the functional component. The shape of the module case may be square or round or irregular, and is adapted to the shape of the functional component, facilitating installation and also reducing the dimensions of the module case during installation.

As a preferred technical means, the functional module is connected into the inner cavity of the module case, including a human body sensing functional component and/or a humidity detection functional component and/or a night-light functional component. The functional modular design prevents functions from being sold in a manner of being integrated with the ventilator body, and customers can select functional modules according to their actual demands. The production cost is saved, the diversity of functions is increased in use, and the flexibility of use is increased.

Compared with the prior art, the utility model has the following beneficial effects.

According to the utility model, the functional component is of an independent modular design: the functional component is independently placed in the module case to become a functional module such that the component can be separated from or assembled with the product and the function can be customized according to demands, thus increasing the diversity of the product function and the flexibility of customer selections. The module case is of a buckle position design, and does not need other connecting parts such as screws during installation. The modular installation is more convenient.

The utility model adopts the common micro-USB as a connecting terminal, and the ventilator body is provided with a plurality of micro-USB receptacles as connecting ports, realizing modular plug-and-pull and ensuring random plug-and-pull. Thus, the utility model is simple in connection operation, ensures plug-and-pull, is easy to identify, avoids mis-connection, and increases safety.

### BRIEF DESCRIPTION OF THE SEVERAL VIEWS OF THE DRAWINGS

FIG. 1 is a structural view of a functional module of the utility model;

FIG. 2 is a structural view of the utility model;

FIG. 3 is a structural view of a connecting wire of the utility model.

### DETAILED DESCRIPTION OF THE INVENTION

To make the objective, technical scheme, and advantages of the utility model clear, the utility model is described in further detail in conjunction with the attached drawings and embodiments. It shall be understood that the specific embodiments described here are not used to limit the utility model, but merely to explain the utility model.

On the contrary, the utility model covers any replacements, modifications, and equivalent methods, as well as schemes defined by claims and made on the basis of the concept and scope of the utility model. Further, in order to ensure that people get a better understanding of the utility model, some specific details are depicted in the following detailed description of the utility model. For those skilled in the art, the utility model can be completely understood without the description of the details.

It needs to be explained that when a component is described as “fixed” at another component, the component can be directly located on the other component or a middle component may exist. When a component is regarded to be “connected” to another component, the component can be directly connected to the other component or a middle component may exist. On the contrary, when it is described that a component is “directly located on” another component, there is no middle component.

Unless otherwise defined, all technical and scientific terms used in this text have meanings usually understood by those skilled in the art that the utility model belongs to. The terms in this text are not used to limit the utility model, but only to describe the objectives of the specific embodiments. The term “and/or” used in this text includes one or more combinations of the relevant listed items.

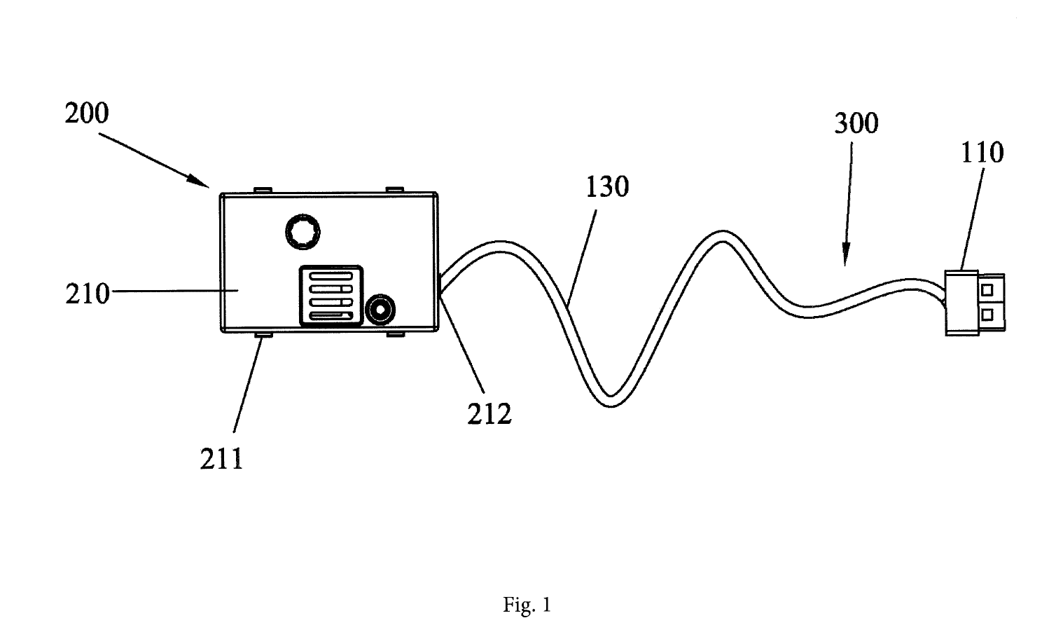

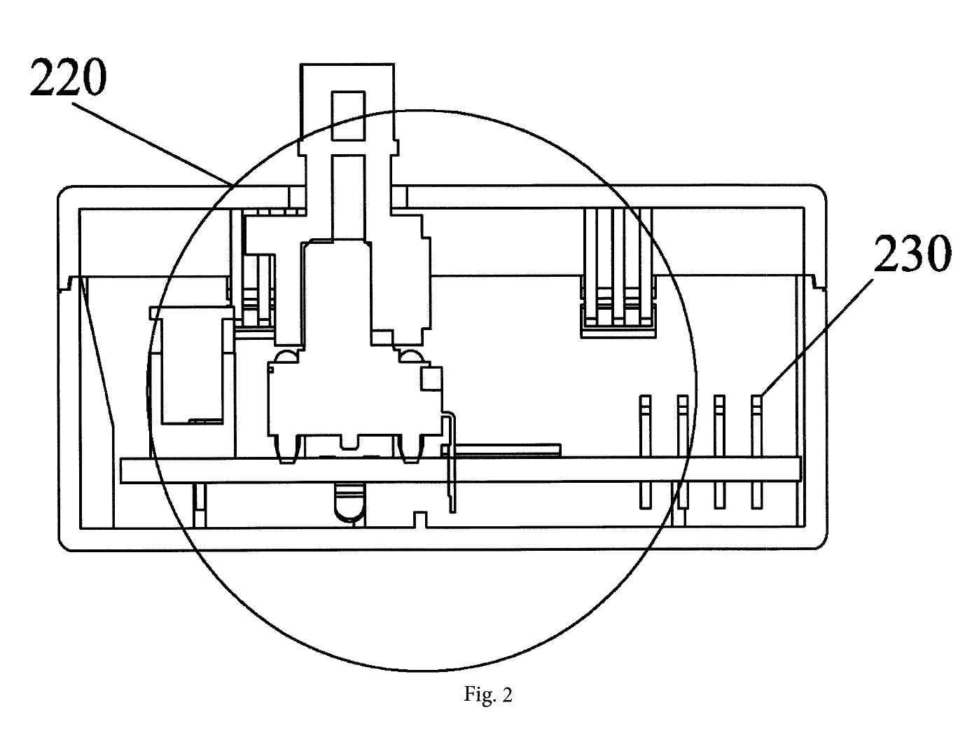

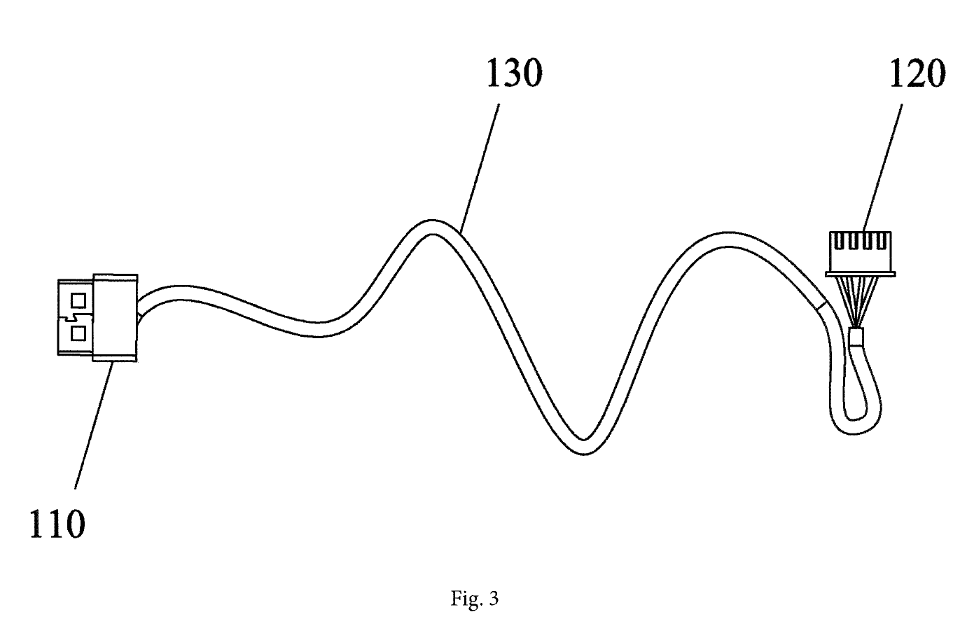

As shown in FIG. 1-3, a functional module 100 of the utility model includes a module case 200 which is capable of accommodating a functional component 220, and a connecting wire 300; the connecting wire 300 includes a common terminal 120, a micro-USB terminal 110 and a round data wire 130 (or wire in other shapes) which connects the common terminal 120 and the micro-USB terminal 110. The functional component 220 is equipped with receptacles 230 corresponding to a common terminal 120, and each one of the receptacles 230 is capable of being fixedly connected with the common terminal 120. The round data wire 130 is provided with a wire clip 131, so that the module case 200 is provided with an outgoing port 212 through which the data wire exits, and the wire clip 131 is placed in the module case 200 to prevent the round data wire 130 from being pulled and falling out of the module case 200 by an external force, increasing the reliability of the use of the connecting wire. The module case 200 is provided with a plurality of fixed buckles 211; the ventilator body (not shown in the drawings) is provided with a plurality of corresponding buckling positions; after the module case 200 is assembled, the module case is buckled with the buckling positions on the ventilator body through the fixed buckles 211 to complete the fixed installation of the module case 200 and the ventilator body. Due to the buckle position structure, connectors such as screws are reduced, time is saved during installation or dismantling, and the installation becomes simpler and more convenient. The ventilator body is provided with a plurality of corresponding micro-USB receptacles (not shown in the drawings), and the connecting wire 300 is provided with a micro-USB terminal 110. To perform connection, it only requires plugging the micro-USB terminal 110 into any one of the micro-USB receptacles, and then a plug-and-play effect is obtained. The use of the micro-USB, which is a common connection approach, brings convenience in identification to users and realizes the plug-and-pull function. The terminal and the receptacles are connected by only one approach, avoiding damage caused by incorrect terminal connection and increasing safety in use. The functional module is connected to the inner cavity of the module case, including a human body sensing functional component and/or a humidity detection functional component and/or a night-light functional component.

The above embodiments are merely preferable embodiments of the utility model, and cannot be regarded as limits to the utility model. Any modifications, equivalent replacement, and improvement made on the basis of the concept and principle of the utility model shall fall within the protective scope of the utility model.

What is claimed is:

1. A ventilator with a functional module, comprising a ventilator body, characterized in that the ventilator body is connected with a functional module; the functional module comprises a module case capable of accommodating a functional component, and a connecting wire; the module case is in a buckled connection with the ventilator body; the ventilator body is provided with a plurality of micro-USB receptacles; one end of the connecting wire is connected with the functional component, and the other end is connected with one of the micro-USB receptacles through a micro-USB terminal.

2. The ventilator with a functional module according to claim 1, wherein the connecting wire is provided with a common terminal which is fixedly connected with an output interface of the functional component, and a data wire capable of transmitting data is connected between the common terminal and the micro-USB terminal.

3. The ventilator with a functional module according to claim 2, wherein the data wire penetrates through a wire clip, and the wire clip is placed in an inner cavity of the module case.

4. The ventilator with a functional module according to claim 1, wherein the module case is provided with a plurality of fixed buckles; the ventilator body is provided with a plurality of corresponding buckling positions; the fixed buckles of the module case and the buckling positions on the ventilator body are mutually fastened to complete the fixed installation of the module case and the ventilator body.

5. The ventilator with a functional module according to claim 4, wherein the module case is of a cavity structure, and has an outline matched with the outline of the functional component.

6. The ventilator with a functional module according to claim 5, wherein the functional module is connected into the inner cavity of the module case, comprising a human body sensing functional element and/or a humidity detection functional element and/or a night-light functional element.
